(19) United States
(12) Patent Application Publication
(10) Pub. No.: US 2026/0354708 A1
(43) Pub. Date: Nov. 12, 2026

(54) MASTICATION DETECTION APPARATUS, MASTICATION DETECTION PROGRAM, MASTICATION DETECTION METHOD, AND GUSTATORY ELECTRIC STIMULATION APPARATUS

(71) Applicants: Ajinomoto Co., Inc., Tokyo (JP); The University of Tokyo, Tokyo (JP)

(72) Inventors: Ryo MATSUMOTO, Kanagawa (JP); Takashi KAKIYAMA, Kanagawa (JP); Takashi SAGISAKA, Tokyo (JP); Yoshinobu OBATA, Tokyo (JP); Amane TAI, Tokyo (JP); Hiromi AOYAMA NAKAMURA, Tokyo (JP)

(73) Assignees: Ajinomoto Co., Inc., Tokyo (JP); The University of Tokyo, Tokyo (JP)

(21) Appl. No.: 19/717,843

(22) Filed: Jun. 24, 2026

Related U.S. Application Data
(63) Continuation of application No. PCT/JP2024/045597, filed on Dec. 24, 2024.

(30) Foreign Application Priority Data
Dec. 25, 2023 (JP) 2023-217732

(51) Int. Cl. A61B 5/11; A61B 5/00; A61N 1/04; A61N 1/05; A61N 1/36

(52) U.S. Cl. CPC A61B 5/1126; A61B 5/0082; A61B 5/6817; A61B 5/7225; A61B 5/7264; A61N 1/0456; A61N 1/0548; A61N 1/36025; A61N 1/36031; A61N 1/36034

(57) ABSTRACT

A mastication detection apparatus includes a sensor to be mounted on a lateral side of a lower jaw and a mastication detection unit that performs detection of a rising zero-cross on a detection signal from the sensor with hysteresis and based on a dead time setting, and when the rising zero-cross is detected, performs comparison of the detection signal from the sensor with a threshold for detecting a mastication motion and detects, based on a result of the comparison, whether a series of motions made by a person wearing the sensor is a mastication motion.

### CROSS-REFERENCE TO RELATED APPLICATION

This application is based upon and claims the benefit of priority from PCT Application PCT/JP2024/045597, filed Dec. 24, 2024, which claims priority from Japanese Patent Application No. 2023-217732, filed Dec. 25, 2023, the entire contents of which are incorporated herein by reference.

### BACKGROUND OF THE INVENTION

### 1. Field of the Invention

The present invention relates to a mastication detection apparatus, a mastication detection program, a mastication detection method, and a gustatory electric stimulation apparatus.

### 2. Description of the Related Art

WO2023/022223 discloses a taste presentation apparatus and the like that can enhance a taste modification effect while suppressing discomfort perceived by the user. Japanese Patent No. 5543929 discloses an input device and the like that are inexpensive and suitable for use in a wearable computer. JP-A-2018-042991 discloses a gustatory electric stimulation apparatus and the like that enable taste adjustment including sustained taste enhancement and the like. JP-A-2008-048791 discloses a mastication count detection apparatus for detecting the number of chews of a human. JP-A-7-171136 discloses a mastication count recording apparatus that records and displays the number of chews. JP-A-2016-093476 discloses a mastication sensation feed device that aims to improve meal satisfaction and enjoyment by providing a simulated chewy texture without causing discomfort perceived by the user even when mastication care foods that lack texture, and that further aims to prevent aspiration, ensure an adequate number of chews, and maintain and improve mastication ability by making the user realize that they are masticating.

### Nonpatent Literature 1

(https://jp.sharp/business/bitescan/) discloses the appearance of a mastication meter with the product name “bitescan”, the capability of visualizing mastication using an application, etc.

However, a technology capable of accurately detecting mastication has not been developed.

### SUMMARY OF THE INVENTION

It is an object of the present invention to at least partially solve the problems in the conventional technology.

The present invention has been achieved in view of the foregoing problem, and it is an object thereof to provide a mastication detection apparatus, a mastication detection program and a mastication detection method capable of accurately detecting mastication, and a gustatory electric stimulation apparatus that is enabled by the accurate mastication detection to apply appropriate electric stimulation with appropriate timing.

To solve the foregoing problem and achieve the object, a mastication detection apparatus according to one aspect of the present invention includes: a sensor to be mounted on a lateral side of a lower jaw; and a mastication detection unit that performs detection of a rising zero-cross on a detection signal from the sensor with hysteresis and based on a dead time setting, and when the rising zero-cross is detected, performs comparison of the detection signal from the sensor with a threshold for detecting a mastication motion and detects, based on a result of the comparison, whether a series of motions made by a person wearing the sensor is a mastication motion.

The mastication detection apparatus according to one aspect of the present invention may further include a filtering unit that performs predetermined filtering processing on the detection signal from the sensor, wherein the mastication detection unit may perform the detection and the comparison based on the detection signal after the predetermined filtering processing is performed.

The predetermined filtering processing may include at least one type of processing among a band-pass filter, a low-pass filter, and direct-current (DC) component removal.

The sensor may be an optical sensor.

The sensor may be worn on an outer ear or a temporomandibular joint region.

The sensor may be worn on an auricle or in an external auditory canal.

The sensor may detect a change in shape of the external auditory canal as a change in distance.

The change in the shape of the external auditory canal may be a continuous one.

The sensor may include a detection element to be disposed at a predetermined position on the outer ear, and an elastic member that is configured to contact the external auditory canal and surrounds the detection element.

The sensor may be a reflective photosensor or a fiber Bragg grating (FBG)-type optical fiber sensor.

A gustatory electric stimulation apparatus according to one aspect of the present invention includes: the mastication detection apparatus according to one aspect of the present invention; a cathode to be mounted on a first site or a second site, the first site being either a back of a head or a dorsal side of a neck of the person, the second site being either an oral cavity or an area around a jaw of the person; an anode to be disposed at the second site of the person when the cathode is mounted on the first site of the person or the first site of the person when the cathode is mounted on the second site of the person; and an electric signal generation unit that applies an electric signal between the anode and the cathode based on a result of the detection by the mastication detection apparatus, wherein the electric signal generation unit outputs a signal having a convex waveform at a predetermined frequency.

The electric signal generation unit may continuously output the electric signal at a specific intensity while the mastication detection apparatus detects that the series of motions made by the person is a mastication motion, and stop outputting the electric signal when the mastication detection apparatus no longer detects that the series of motions made by the person is a mastication motion.

The electric signal generation unit may stop output of the electric signal with a gradient.

The electric signal generation unit may output an electric signal having a specific convex waveform when a number of times the mastication detection apparatus detects that the series of motions made by the person is a mastication motion reaches a predetermined number, and output the electric signal having the specific convex waveform again when the number of times the mastication detection apparatus detects that the series of motions made by the person is a mastication motion reaches the predetermined number after the output.

The electric signal generation unit may control output of the electric signal with a gradient.

A mastication detection program according to one aspect of the present invention is one for causing a computer connected to a sensor mounted on a lateral side of a lower jaw to function as a mastication detection unit that performs detection of a rising zero-cross on a detection signal from the sensor with hysteresis and based on a dead time setting, and when the zero-cross is detected, performs comparison of the detection signal from the sensor with a threshold for detecting a mastication motion and detects whether a series of motions made by a person wearing the sensor is a mastication motion based on a result of the comparison.

A mastication detection method according to one aspect of the present invention is one wherein a computer connected to a sensor mounted on a lateral side of a lower jaw performs a mastication detection step of performing detection of a rising zero-cross on a detection signal from the sensor with hysteresis and based on a dead time setting, and when the zero-cross is detected, performing comparison of the detection signal from the sensor with a threshold for detecting a mastication motion and detecting whether a series of motions made by a person wearing the sensor is a mastication motion based on a result of the comparison.

According to the present invention, an effect that mastication can be accurately detected is provided. Moreover, according to the present invention, an effect that appropriate electric stimulation can be applied with appropriate timing by virtue of accurate mastication detection is provided.

The above and other objects, features, advantages and technical and industrial significance of this invention will be better understood by reading the following detailed description of presently preferred embodiments of the invention, when considered in connection with the accompanying drawings.

### BRIEF DESCRIPTION OF THE DRAWINGS

FIG. 1 is a block diagram of an example of a configuration of a gustatory electric stimulation apparatus 1;

FIG. 2 is a diagram of a configuration and appearance of a wearable headset in which the gustatory electric stimulation apparatus 1 is implemented;

FIG. 3 is a diagram of a use state of the wearable headset shown in FIG. 2;

FIG. 4 is a diagram mainly of a detection example of a mastication motion;

FIG. 5 is a diagram mainly of a first presentation example of a current; and

FIG. 6 is a diagram mainly of a second presentation example of the current.

### DETAILED DESCRIPTION OF THE PREFERRED EMBODIMENTS

An embodiment of a mastication detection apparatus, a mastication detection program, a mastication detection method, and a gustatory electric stimulation apparatus according to the present invention will be explained in detail below with reference to the drawings. The present invention is not limited by the present embodiment. The present invention is not intended to be limited to the configurations described in the embodiment and drawings explained below, and also encompasses configurations equivalent thereto. In particular, in the present embodiment, an example where a mastication detection apparatus is incorporated in a gustatory electric stimulation apparatus will be described. However, the present invention is not limited to this example.

### 1. Overview of Gustatory Electric Stimulation

Electric stimulation to the perioral region has attracted attention in recent years as a next-generation salt reduction technology. A gustatory electric stimulation apparatus that applies electric stimulation to the perioral region may be able to be used as a “new seasoning” that achieves further salt reduction and enhances food palatability.

However, conventional gustatory electric stimulation apparatuses need the user to manually turn on the electric stimulation switch each time the user brings food to the mouth, and such apparatuses are difficult to use from a convenience standpoint. Moreover, with these conventional gustatory electric stimulation apparatuses, the duration of electric stimulation (for example, 30 seconds) is set in advance, and electric stimulation is applied for that duration once the switch is turned on. Electric current thus continues to flow to the user even after the user has completed mastication and swallowing, possibly resulting in the user experiencing a sense of discomfort or unpleasantness.

In the present embodiment, mastication is thus detected to enable application of appropriate electric stimulation to the user with appropriate timing. Specifically, according to the present embodiment, the mastication and swallowing of the user is detected and the electric stimulation is automatically controlled to be turned on or off. Such a manner of electric stimulation can improve convenience and reduce the risk of the user experiencing unnecessary electric stimulation.

### 2. Configuration

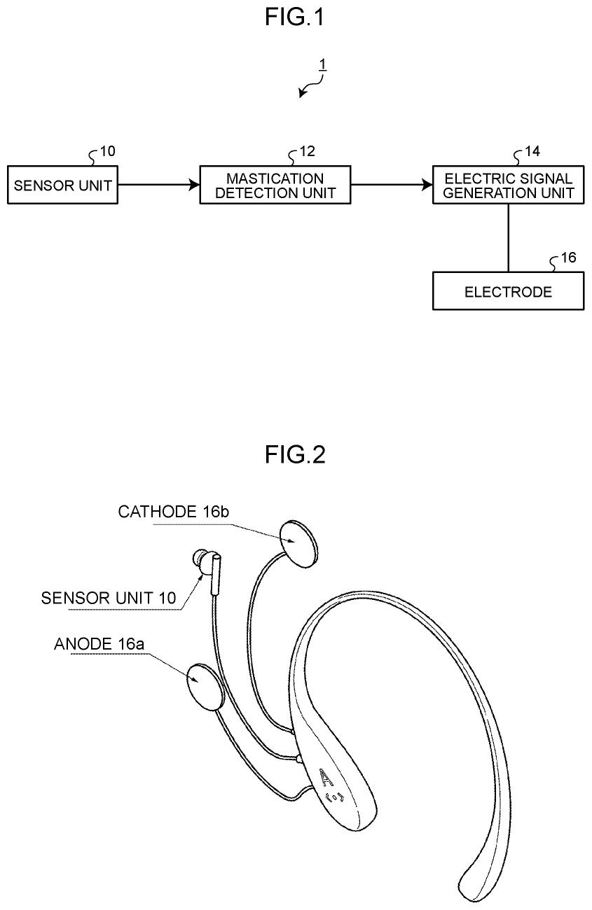

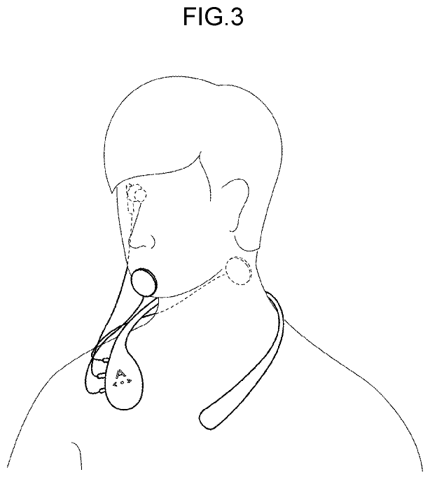

FIG. 1 is a block diagram of an example of a configuration of a gustatory electric stimulation apparatus 1. The gustatory electric stimulation apparatus 1 includes a sensor unit 10, a mastication detection unit 12 (in other words, the mastication detection apparatus according to the present invention), an electric signal generation unit 14, and electrodes 16 (an anode 16a and a cathode 16b). Examples of the types of taste that can be modified by the gustatory electric stimulation apparatus 1 include basic tastes (sweetness, sourness, saltiness, bitterness, and umami), richness, fattiness, and spiciness. For reference, FIG. 2 depicts the configuration and appearance of a wearable headset (in other words, an example of a wearable computer) in which the gustatory electric stimulation apparatus 1 is implemented. FIG. 3 depicts the wearable headset in use.

### 2-1. Configuration of Sensor Unit (see FIGS. 1 and 2)

The sensor unit 10 is mounted on a body part (for example, a lateral side of the lower jaw such as the outer ear [auricle or external auditory canal] or the temporomandibular joint region). For example, the sensor unit 10 may be mounted on a natural orifice. As employed herein, a “natural orifice of the body” refers to an opening of an organ or the like formed in the body. Examples include the outer ear (external auditory canal), nasal cavity, oral cavity, anus, and vagina. According to the present embodiment, the outer ear (including the external auditory canal) in particular is used as a natural orifice of the body. While various types of sensor units 10 may be used, an optical sensor (such as a reflective photosensor or an FBG-type optical sensor) capable of optically measuring a distance to a measurement target is preferably employed. The optical sensor includes a light-emitting element such as a light-emitting diode (LED) or a light bulb, and a light-receiving element such as a phototransistor, a photodiode, or a charge-coupled device (CCD). If an infrared LED is used as the LED, one that emits infrared rays at a wavelength that does not penetrate the human body needs to be selected. In the present embodiment, the sensor unit 10 will hereinafter be explained as an optical sensor. As for the shape of the sensor unit 10, an “over-ear type” or an “insertion type” is suitable for ease of use.

### Over-Ear Type

The sensor unit 10 of the over-ear type is mounted to cover the outer ear from the outside practically without being inserted into the external auditory canal. Once the sensor unit 10 is mounted, the relative position of the sensor unit 10 to the external auditory canal is substantially fixed. Meanwhile, the shape of the external auditory canal changes differently depending on location. For example, a region that changes in shape in response to movement of the tongue differs from a region that changes in shape in response to movement of the eyeball. The mounting direction of the sensor unit 10 with respect to the outer ear is therefore desirably adjusted as appropriate depending on the motion to be measured. Here, the direction of the entire sensor unit 10 may be adjusted, or the direction of only a detection element may be adjusted.

The sensor unit 10 includes an emitting element and a receiving element serving as the detection element. Part of the measurement light emitted from the emitting element is reflected at the surface of the external auditory canal and returns to the receiving element. If the outer ear of the wearer of the sensor unit 10 moves, the distance from the detection element of the sensor unit 10 to the reflecting surface of the external auditory canal changes. As a result, the intensity of the reflected light incident on the detection element (receiving element) changes. By monitoring changes in the intensity of this reflected light, changes in the shape of the external auditory canal can thus be detected. A plurality of detection elements may be provided as needed. Examples of the emitting element include light-emitting elements such as an LED and a light bulb. Examples of the receiving element include light-receiving elements such as a phototransistor, a photodiode, and a CCD. In particular, when an LED is employed as the emitting element and a phototransistor or photodiode is employed as the receiving element, noise in the detection signal can be reduced, and a high-quality detection signal can thus be obtained.

### Insertion Type

In the insertion type, the emitting element and the receiving element serving as the detection element are structured to be surrounded by an elastic member. The elastic member contacts the surface of the external auditory canal when the sensor unit 10 is inserted into the external auditory canal. Thus, even after the sensor unit 10 is inserted into a predetermined position in the external auditory canal, the detection element can move within the external auditory canal of the wearer as the elastic member deforms. Meanwhile, the tympanic membrane of the wearer does not move significantly even when the shape of the external auditory canal changes. The detection element can thus slightly move relative to the tympanic membrane.

As described above, the shape of the external auditory canal changes differently depending on location. With the shape and hardness of the elastic member appropriately adjusted, the detection element of the sensor unit 10 can easily detect changes in the shape of a specific part of the external auditory canal. Such detection enables the sensor unit 10 to respond only to a specific motion of the wearer. It is also effective to combine a relatively hard member and a relatively soft member as the elastic member. Such an elastic member enables even finer adjustment.

Part of the measurement light emitted from the emission element is reflected at the surface of the tympanic membrane and returns to the receiving element. If the shape of the external auditory canal changes, the elastic member deforms to cause a slight relative movement of the detection element, and the distance from the detection element to the reflecting surface of the tympanic membrane changes. As a result, the intensity of the reflected light incident on the detection element (receiving element) changes. By monitoring changes in the intensity of this reflected light, changes in the shape of the external auditory canal can thus be detected. A plurality of detection elements may be provided as needed.

The sensor unit of the insertion type can amplify a detection signal, to be described below, by using the foregoing relative movement of the detection element. This amplification by the relative movement is not electrical but attributable to the mechanism of the sensor unit 10. The amplified signal is thus a high-quality signal with an excellent S/N ratio.

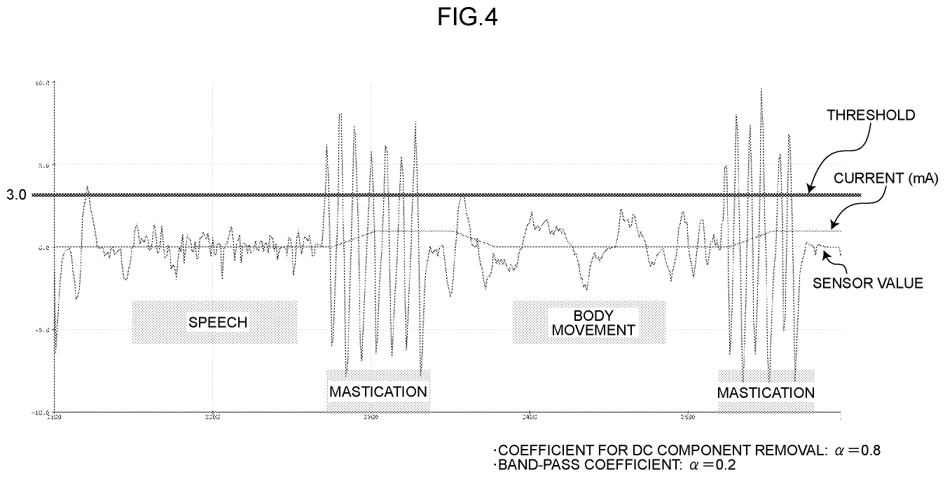

### 2-2. Configuration of Mastication Detection Unit (See FIG. 4)

The mastication detection unit 12 can be implemented by a computer, for example. When the detection element (receiving element) of the sensor unit 10 receives reflected light, the sensor unit 10 generates a detection signal based on the detection intensity, and transmits this to the mastication detection unit 12. When the mastication detection unit 12 receives the detection signal from the sensor unit 10, the mastication detection unit 12 performs detection of a rising zero-cross on the detection signal with hysteresis and based on a setting of a dead time (for example, 300 ms). When the rising zero-cross is detected, the mastication detection unit 12 compares the detection signal with a threshold for detecting a mastication motion, and detects whether a series of motions (hereinafter, referred to as a “continuous motion”) made by the person wearing the sensor unit 10 (hereinafter, referring to as a “wearer”) is a mastication motion based on the comparison result. The mastication detection unit 12 may include a filtering unit that performs predetermined filtering processing (for example, at least one type of processing among a band-pass filter, a low-pass filter, and DC component removal) on the detection signal. When the mastication detection unit 12 includes the filtering unit, the mastication detection unit 12 performs the detection of a rising zero-cross and the comparison of the detection signal with the threshold (see FIG. 4) based on the detection signal after the predetermined filtering processing.

In the present embodiment, the dead time is set so that only waveforms appearing to be a mastication rhythm are responded to. For mastication motion detection, the mastication detection unit 12 may be configured to detect that the continuous motion is a mastication motion if the detection signal exceeds a threshold (see FIG. 4). For the filtering processing, the band-pass filter and/or low-pass filter is preferably applied within a range where waveform characteristics are not lost. For example, the band-pass (low-pass) coefficient is desirably set to a value in the range of, e.g., 0.2 to 0.4 based on the actually measured detection signal (sensor value). To prevent misdetection where a continuous motion that is actually a motion other than a mastication motion (such as speech, body movement, pushing of the sensor unit 10 into the outer ear, and removal of the sensor unit 10 from the outer ear) is detected as a mastication motion, a low-pass filter may be applied more strongly, or the waveform may be focused on, for example.

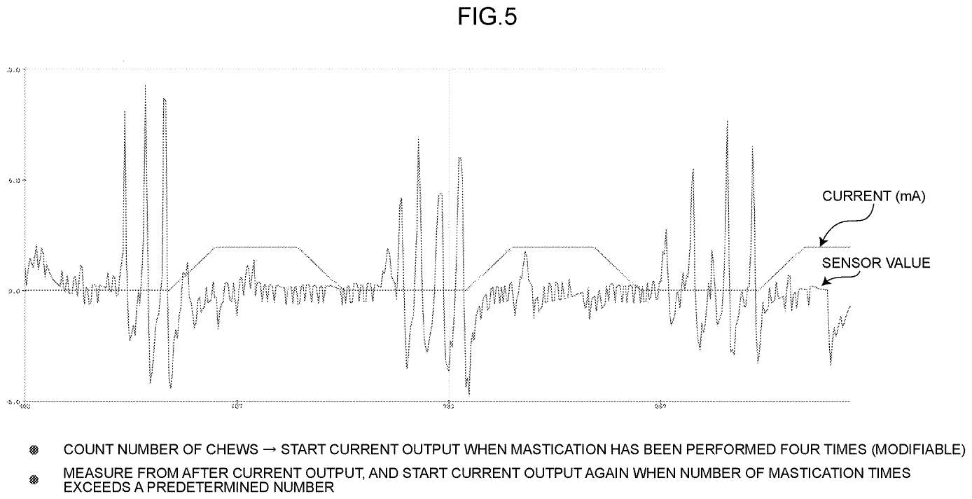

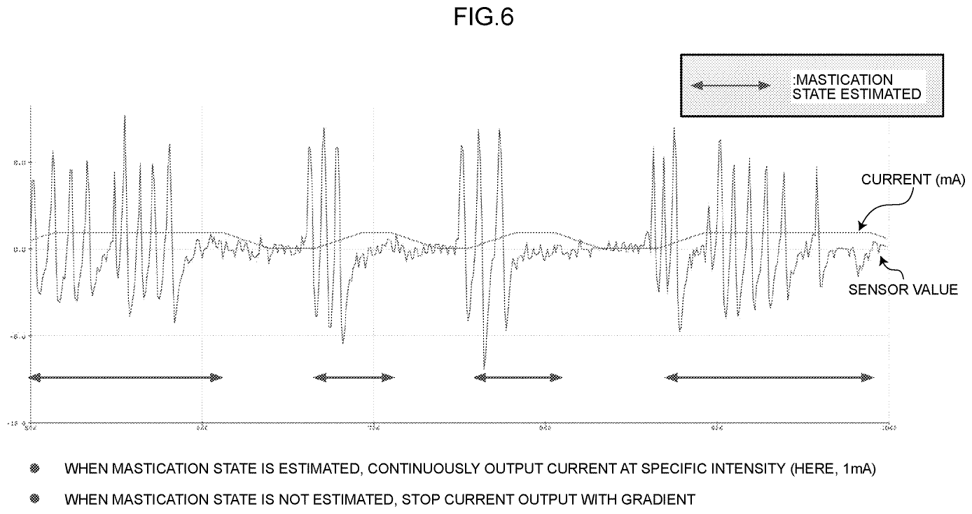

### 2-3. Configuration of Electric Signal Generation Unit (See FIGS. 5 and 6)

The electric signal generation unit 14 can be implemented by, for example, a boost circuit, an operational amplifier, and peripheral circuits serving as a current control unit, and a current sensor integrated circuit (IC) and peripheral circuits serving as a current measurement unit. The electric signal generation unit 14 applies an electric signal between the anode 16a and the cathode 16b based on the result of the detection by the mastication detection unit 12. The electric signal generation unit 14 outputs a signal having a convex waveform at a predetermined frequency.

The electric signal generation unit 14 may output an electric signal having a specific convex waveform when the number of times the mastication detection unit 12 detects that a continuous motion is a mastication motion reaches a predetermined number (for example, four). After the output, the electric signal having the specific convex waveform is outputted again when the number of times the mastication detection unit 12 detects that a continuous motion is a mastication motion reaches the predetermined number (for example, four) (see FIG. 5). The electric signal generation unit 14 may control the output of the electric signal with a gradient. By controlling the output of the electric signal with a gradient, intraocular phosphene caused by the electric stimulation in the user can be suppressed, and consequently the usability of the gustatory electric stimulation apparatus 1 can be enhanced.

Alternatively, the electric signal generation unit 14 may continuously output the electric signal at a specific intensity while the mastication detection unit 12 detects that a continuous motion is a mastication motion, and stop outputting the electric signal when the mastication detection unit 12 no longer detects that a continuous motion is a mastication motion (see FIG. 6). The electric signal generation unit 14 may stop the output of the electric signal with a gradient. By stopping the output of the electric signal with a gradient, intraocular phosphene caused by the electric stimulation in the user can be suppressed, and consequently the usability of the gustatory electric stimulation apparatus 1 can be enhanced.

### 2-4. Configuration of Electrodes (See FIGS. 1, 2, and 3)

The electrodes 16 include: the anode 16a to be disposed at either the back of the head or the dorsal side of the neck of the wearer (first site) or either the oral cavity or the area around the jaw of the wearer (second site); and the cathode 16b to be mounted on the second site of the wearer when the anode 16a is disposed at the first site of the wearer or to be mounted on the first site of the wearer when the anode 16a is disposed at the second site of the wearer.

According to the gustatory electric stimulation apparatus 1 configured as described above, the sensor unit 10 is mounted on a lateral side of the lower jaw of the body (in the present embodiment, the external auditory canal). Changes in the shape of the lateral side of the lower jaw can thus be detected in an environment where disturbance factors such as external light are excluded as much as possible. As a result, a highly reliable detection signal can be stably obtained from the sensor unit 10. Based on the obtained detection signal, the mastication motion can be accurately detected, and appropriate electric stimulation can be applied to the wearer with appropriate timing.

The sensor unit 10 is not limited to being an optical sensor. Other sensors may be applied as the sensor unit 10 as long as the sensors can be mounted on a lateral side of the lower jaw of the body and detect changes in the shape of the lateral side of the lower jaw.

For example, the sensor unit 10 may be an acoustic sensor. The sensor unit 10 includes an emitting element and a receiving element serving as a detection element. Examples of the acoustic sensor include an ultrasonic distance sensor. Examples of the emitting element include a speaker. Examples of the receiving element include a microphone. Some of the measurement waves emitted from the emitting element are reflected at the surface of the external auditory canal and return to the receiving element. If the outer ear of the wearer moves, the distance from the detection element of the sensor unit 10 to the reflecting surface of the external auditory canal changes. As a result, the amount of time that elapses before the reflected waves are incident on the detection element (receiving element) changes. By monitoring such changes in time, changes in the shape of the external auditory canal can thus be detected. The frequency of the sound waves used as the measurement waves can be 20 kHz or higher, which is undetectable with human hearing, for example. Alternatively, the sensor unit 10 may be one that can detect mastication sound (for example, see JP-A-2019-047859 or JP-A-2012-196284).

For example, the sensor unit 10 may be an FBG-type optical fiber sensor. With the FBG-type optical fiber sensor, a certain structural change can be induced within the optical fiber, and a specific wavelength passing through the optical fiber can be captured by utilizing the structural change as a diffraction grating. The optical fiber of the FBG-type optical fiber sensor is laid along the surface of the external auditory canal so that the optical fiber deforms to follow movement of the outer ear. Since the wavelength of the light passing through the optical fiber changes as the optical fiber deforms, changes in the external auditory canal associated with the movement of the outer ear can be detected by monitoring changes in the wavelength.

When an acoustic sensor or an optical sensor as in the present embodiment is used as the sensor unit 10, unlike a pressure sensor, the sensor unit 10 itself does not need to be strongly pressed into a natural orifice of the body. Discomfort perceived by the wearer can thus be reduced. This enables long-term wearing, and the sensor can be suitably used as the sensor unit 10.

The use of an optical sensor as the sensor unit 10 also has the advantage that the sensor unit 10 can be simplified in structure, and constructed at a lower cost, compared with pressure sensors and the like.

The gustatory electric stimulation apparatus 1 can also be used to improve the quality of life (QOL) of people who need to limit excessive salt intake, like patients with chronic kidney disease or hypertension. Specifically, the gustatory electric stimulation apparatus 1 can be used to alleviate the anxiety about when symptoms may worsen, which is felt due to inability to reduce salt intake. The gustatory electric stimulation apparatus 1 can also be used to create the enjoyment of wanting to occasionally eat something heavily salty.

The gustatory electric stimulation apparatus 1 can also be used to support lifestyle improvement utilizing mastication information. Specifically, the gustatory electric stimulation apparatus 1 can visualize the number of chews, mastication intensity, and mastication time, which enables real-time monitoring of, e.g., how many times one chews per bite, the level of mastication force, and the like. The gustatory electric stimulation apparatus 1 may adjust the intensity, frequency, or duration of the electric stimulation, the type of taste to be given, or the like based on mastication conditions such as the number of chews, mastication intensity, and mastication time. There are reports that fewer chews per bite result in less stimulation of the satiety center, leading to increased food intake and a tendency toward obesity, and that a decrease in mastication force increases the risk of lifestyle-related diseases. For example, with the gustatory electric stimulation apparatus 1 made IoT-enabled and linked with a smartphone, the user's own mastication status can be visualized. This facilitates the user improving lifestyle habits through mastication. Utilization for improving the dietary behavior of people who need to limit excessive salt intake, such as patients with chronic kidney disease or hypertension, is also conceivable.

Mastication information obtained by the gustatory electric stimulation apparatus 1 can be used in virtual space. The gustatory electric stimulation apparatus 1 may be able to be integrated in the future with metaverse-related devices such as virtual reality (VR) devices, and used for taste adjustment and taste creation of digital foods consumed in virtual space. If the taste changing and mastication detection functions are successfully integrated with metaverse-related devices, the user will be able to not only digitize the hardness of foods being consumed in virtual space to adjust the texture of digital foods, but also freely adjust the taste of those digital foods with a high degree of freedom through electric stimulation. Application to other sensory and physical stimuli such as smell, warm/cold air, and ultrasonic waves is also possible.

Although the invention has been described with respect to specific embodiments for a complete and clear disclosure, the appended claims are not to be thus limited but are to be construed as embodying all modifications and alternative constructions that may occur to one skilled in the art that fairly fall within the basic teaching herein set forth.

What is claimed is:

1. A mastication detection apparatus comprising:
a sensor to be mounted on a lateral side of a lower jaw; and
a mastication detection unit that performs detection of a rising zero-cross on a detection signal from the sensor with hysteresis and based on a dead time setting, and when the rising zero-cross is detected, performs comparison of the detection signal from the sensor with a threshold for detecting a mastication motion and detects, based on a result of the comparison, whether a series of motions made by a person wearing the sensor is a mastication motion.

2. The mastication detection apparatus according to claim 1, further comprising a filtering unit that performs predetermined filtering processing on the detection signal from the sensor, wherein
the mastication detection unit performs the detection and the comparison based on the detection signal after the predetermined filtering processing is performed.

3. The mastication detection apparatus according to claim 2, wherein the predetermined filtering processing includes at least one type of processing among a band-pass filter, a low-pass filter, and direct-current (DC) component removal.

4. The mastication detection apparatus according to claim 3, wherein the sensor is an optical sensor.

5. The mastication detection apparatus according to claim 4, wherein the sensor is worn on an outer ear or a temporomandibular joint region.

6. The mastication detection apparatus according to claim 5, wherein the sensor is worn on an auricle or in an external auditory canal.

7. The mastication detection apparatus according to claim 6, wherein the sensor detects a change in shape of the external auditory canal as a change in distance.

8. The mastication detection apparatus according to claim 7, wherein the change in the shape of the external auditory canal is a continuous one.

9. The mastication detection apparatus according to claim 8, wherein the sensor includes a detection element to be disposed at a predetermined position on the outer ear, and an elastic member that is configured to contact the external auditory canal and surrounds the detection element.

10. The mastication detection apparatus according to claim 9, wherein the sensor is a reflective photosensor or a fiber Bragg grating (FBG)-type optical fiber sensor.

11. A gustatory electric stimulation apparatus comprising:
the mastication detection apparatus according to claim 1;
an anode to be disposed at a first site or a second site, the first site being either a back of a head or a dorsal side of a neck of the person, the second site being either an oral cavity or an area around a jaw of the person;
a cathode to be mounted on the second site of the person when the anode is disposed at the first site of the person or the first site of the person when the anode is disposed at the second site of the person; and
an electric signal generation unit that applies an electric signal between the anode and the cathode based on a result of the detection by the mastication detection apparatus, wherein
the electric signal generation unit outputs a signal having a convex waveform at a predetermined frequency.

12. The gustatory electric stimulation apparatus according to claim 11, wherein the electric signal generation unit continuously outputs the electric signal at a specific intensity while the mastication detection apparatus detects that the series of motions made by the person is a mastication motion, and stops outputting the electric signal when the mastication detection apparatus no longer detects that the series of motions made by the person is a mastication motion.

13. The gustatory electric stimulation apparatus according to claim 12, wherein the electric signal generation unit stops output of the electric signal with a gradient.

14. The gustatory electric stimulation apparatus according to claim 11, wherein the electric signal generation unit outputs an electric signal having a specific convex waveform when a number of times the mastication detection apparatus detects that the series of motions made by the person is a mastication motion reaches a predetermined number, and outputs the electric signal having the specific convex waveform again when the number of times the mastication detection apparatus detects that the series of motions made by the person is a mastication motion reaches the predetermined number after the output.

15. The gustatory electric stimulation apparatus according to claim 14, wherein the electric signal generation unit controls output of the electric signal with a gradient.

16. A mastication detection program for causing a computer connected to a sensor mounted on a lateral side of a lower jaw to function as a mastication detection unit that performs detection of a rising zero-cross on a detection signal from the sensor with hysteresis and based on a dead time setting, and when the zero-cross is detected, performs comparison of the detection signal from the sensor with a threshold for detecting a mastication motion and detects whether a series of motions made by a person wearing the sensor is a mastication motion based on a result of the comparison.

17. A mastication detection method wherein
a computer connected to a sensor mounted on a lateral side of a lower jaw performs
a mastication detection step of performing detection of a rising zero-cross on a detection signal from the sensor with hysteresis and based on a dead time setting, and when the zero-cross is detected, performing comparison of the detection signal from the sensor with a threshold for detecting a mastication motion and detecting whether a series of motions made by a person wearing the sensor is a mastication motion based on a result of the comparison.
